(19) United States
(12) Patent Application Publication
(10) Pub. No.: US 2018/0353771 A1
(43) Pub. Date: Dec. 13, 2018

(54) FLEXIBLE PHOTOTHERAPY DEVICE FOR WOUND TREATMENT

(71) Applicant: SABIC Global Technologies B.V., Bergen op Zoom (NL)

(72) Inventors: Sang Hoon KIM, Seoul (KR); Eun-Uk KIM, Gyeonggi-do (KR); Sang-Jin KIM, Seoul (KR)

(21) Appl. No.: 15/780,720

(22) PCT Filed: Dec. 2, 2016
(86) PCT No.: PCT/IB2016/057322

§ 371 Date: Jun. 1, 2018

Related U.S. Application Data
(60) Provisional application No. 62/262,592, filed on Dec. 3, 2015.

(51) Int. Cl. A61N 5/06

(52) U.S. Cl. CPC A61N 5/0616; A61N 2005/0653; A61N 2005/0665; A61N 2005/0663; A61N 2005/0645

(57) ABSTRACT

The present disclosure is directed to wearable phototherapy devices; and more particularly, flexible, attachable, phototherapy devices for wound treatment at a skin surface of a user. The flexible devices include a flexible attachment strip configured to contact and secure the device to the skin surface, a moistening band attached to a bottom surface of the attachment strip and configured to contact the skin surface, and a light-emitting assembly including at least one organic light-emitting diode (OLED) and at least one emission modifier. The flexible device can further include one or more sensors disposed along the bottom surface of the flexible attachment strip, a near field communication (NFC) antenna, and a flexible printed circuit board (FBCB) including a communication microchip and a memory microchip connected to the flexible attachment strip such that the flexible device can provide remote medical treatment.

### TECHNICAL FILED

This disclosure is directed to wearable, flexible medical devices for phototherapy wound treatment.

### BACKGROUND

Phototherapy treatments are known to be effective in assisting the healing of skin and tissue in and around a wound site. Phototherapy can affect human skin because light can be absorbed, reflected, and scattered in the human skin. Additionally, light from phototherapy can promote biochemical reactions in skin cells, enhance collagen production, as well as speed the repair of damaged skin tissue and skin regeneration.

However, phototherapy treatment alone is typically not as effective for serious or complex skin wounds involving deep punctures or tears, surgical sites, dermal burn, etc. Commonly, these wounds require an enhanced level of treatment, such as for example, additional applications of topical medication, or specialized dressings or wound coverings. Monitoring of the healing process, as well as modifying the treatment is also difficult.

Existing commercially available phototherapy systems typically utilize light sources such as LEDs, fluorescent lamps, halogen lamps, and ultraviolet lamps. These systems are not usually directly applicable to the wound site because the light source can generate too much heat, and also can be in a rigid structure that cannot be effectively or comfortably attached to the skin.

Thus, there is a need for improvement in phototherapy devices for treatment of skin wounds.

### SUMMARY

The present disclosure is directed to wearable phototherapy devices; and more particularly, flexible, attachable, phototherapy devices for wound treatment at a skin surface of a user. According to one embodiment of the present disclosure, the flexible devices include a flexible attachment strip configured to contact and secure the device to the skin surface, a moistening band attached to a bottom surface of the attachment strip and configured to contact the skin surface, and a light-emitting assembly including at least one organic light-emitting diode (OLED) and at least one emission modifier. The flexible attachment strip can have a bottom surface facing the skin surface and an oppositely disposed top surface. The light-emitting assembly can have a bottom surface facing the skin surface and an oppositely disposed top surface, the light emitting assembly disposed between the top surface of the moistening band and the bottom surface of the attachments strip.

According to a further embodiment of the present disclosure, the flexible device can further provide monitoring of the wound area and can include one or more sensors disposed along the bottom surface of the flexible attachment strip, a near field communication (NFC) antenna connected to the flexible attachment strip, a battery connected to the flexible attachment strip, and a flexible printed circuit board (F/PCB), including a communication microchip and a memory microchip, connected to the flexible attachment strip.

The present disclosure is also directed to methods of phototherapeutic treatment of a wound utilizing the flexible devices disclosed herein. According to one embodiment, the method can include determining a treatment protocol, attaching the flexible device to a surface of the skin, and activating the light-emitting assembly to emit light having a wavelength range and intensity according to the treatment protocol.

According to another embodiment, methods of phototherapeutic treatment of a wound utilizing the flexible devices disclosed herein can include determining a first treatment protocol, attaching the flexible device to a surface of the skin, activating the light-emitting assembly to emit light having a wavelength range and intensity according to the first treatment protocol, acquiring treatment data of the first protocol through the one or more sensors, transmitting the treatment data from the one or more sensors of the device to an external terminal, comparing treatment data from the first protocol treatment with a treatment database, determining an adjustment from the first treatment protocol to a second treatment protocol, transmitting instructions from the external terminal to the communication microchip through the antenna of the device for the second protocol, and activating the light-emitting assembly to emit light having a wavelength range and intensity according to the second treatment protocol.

### BRIEF DESCRIPTION OF THE DRAWINGS

The foregoing summary, as well as the following detailed description of preferred embodiments of the application, will be better understood when read in conjunction with the appended drawings. For the purposes of illustrating the flexible wearable phototherapy devices of the present application, there is shown in the drawings preferred embodiments. It should be understood, however, that the application is not limited to the precise arrangements shown. In the drawings:

FIG. 1A is a bottom view of a flexible phototherapy device according to one embodiment of the present disclosure;

FIG. 1B is a top view of the flexible phototherapy device illustrated in FIG. 1A;

FIG. 1C is an exploded side view of the flexible phototherapy device illustrated FIGS. 1A-B;

FIG. 2 is a schematic side view representation of a light-emitting assembly according to one embodiment, including a thin film encapsulant, an OLED including an emissive layer disposed between electrodes, and a substrate material on the bottom surface;

FIG. 3 is a schematic perspective view representation of the light-emitting assembly of FIG. 2 without the thin film encapsulant shown;

FIG. 4 is schematic side view representation of a light-emitting assembly according to another embodiment, including an OLED having an emissive layer, an electron transport layer, a hole transport layer, and a hole injection layer, disposed between the electrodes;

FIG. 5 is a schematic side view representation of a light-emitting assembly according to another embodiment, including an OLED having a blue emissive layer, and a red Color Conversion Layer (CCL) disposed between the anode and the substrate;

FIG. 6 is a schematic side view representation of a light-emitting assembly according to another embodiment, including different emission modifiers disposed below the OLED; and

FIG. 7 is a schematic side view representation of a light emitting assembly according to another embodiment including an OLED having a blue emissive layer and a red and green CCL layer.

### DETAILED DESCRIPTION

In this document, the terms “the” “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. It is also to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination.

When a range of values is expressed, another embodiment includes from the one particular value and/or to the other particular value. Similarly, when values are expressed as approximations, by use of the antecedent “about,” it will be understood that the particular value forms another embodiment. The modifier “about” used in connection with a quantity is inclusive of the stated value and has the meaning dictated by the context (e.g., it includes the degree of error associated with measurement of the particular quantity based upon the instrumentation or methodology used to obtain the data). All ranges disclosed herein are inclusive of the endpoints, and the endpoints are independently combinable with each other. Further, reference to values stated in ranges includes each and every value within that range. For example, if a range is disclosed having a first endpoint 10, and second endpoint 15, then 11, 12, 13, and 14 are also disclosed.

As used herein, the term “light” means electromagnetic radiation including ultraviolet, visible or infrared radiation.

As used herein, the term “transparent” means that the level of transmittance for a disclosed composition is greater than 50%. In some embodiments, the transmittance can be at least 60%, 70%, 80%, 85%, 90%, or 95%, or any range of transmittance values derived from the above exemplified values. In the definition of “transparent”, the term “transmittance” refers to the amount of incident light that passes through a sample measured in accordance with ASTM D1003 at a thickness of 3.2 millimeters. Unless specified to the contrary herein, all test standards are the most recent standard in effect at the time of filing this application.

As used herein, an “emission modifier” is a composition or structure that can modify the light emission properties of the light emitted from the source OLED. Such properties can include, but are not limited to, transmission intensity, wavelength transmission, and range of transmitted wavelengths.

For purposes of this disclosure, the terms “bottom” and “below,” as well as any derivatives thereof, are intended to define the side or surface of any disclosed structure, or the relative position of a structure, that is in a direction facing, or configured to face, the skin surface. Conversely, the terms “top” and “above,” as well as any derivatives thereof, are intended to define the corresponding opposing side or surface of the disclosed structure, or the opposing relative position with respect to the skin surface.

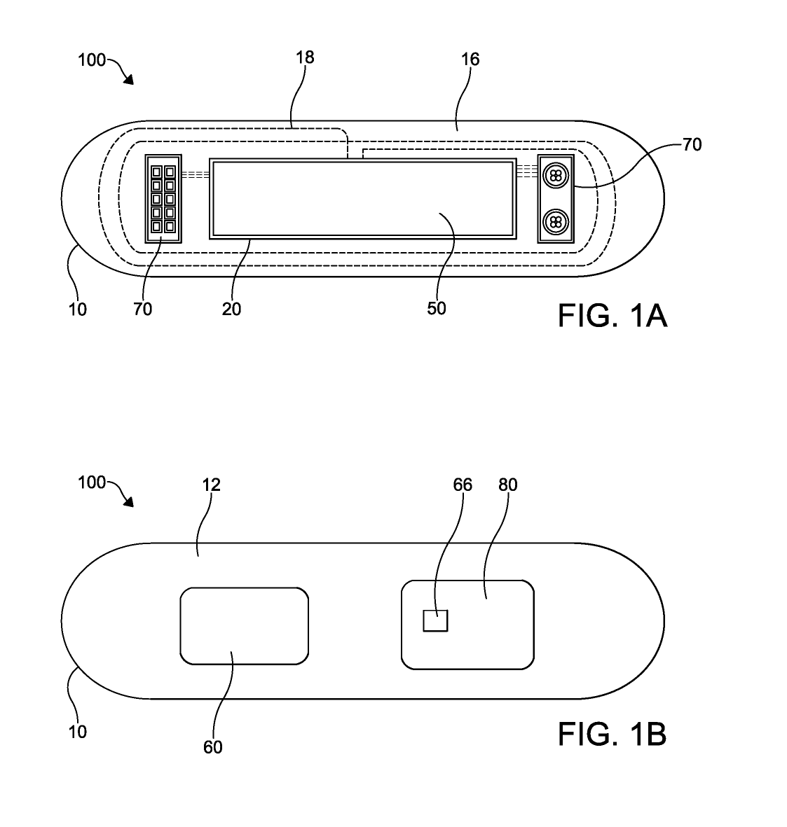

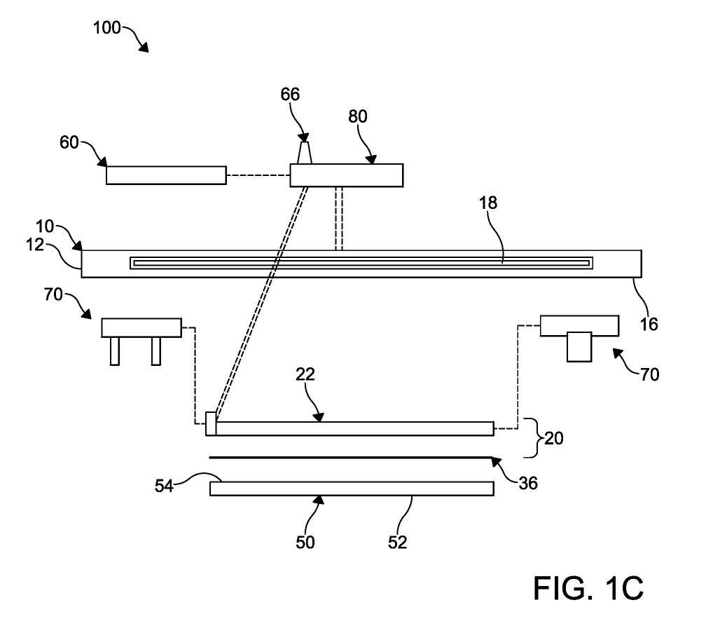

Referring to FIG. 1A-C, according to one embodiment of the present disclosure, a flexible device 100 is shown. The flexible device 100 includes a flexible attachment strip 10 configured to contact and secure the device to the skin surface, and can have a bottom surface 16 facing the skin surface and an oppositely disposed top surface 12. The flexible device 100 further includes a moistening band 50 having a bottom surface 52 facing the skin surface and an oppositely disposed top surface 54, the moistening band 50 attached to the bottom surface 16 of the attachment strip 10 and configured to contact the skin surface.

According to one embodiment, the flexible attachment strip 10 is a standard medical dressing known in the art. In a further embodiment, the strip has permeability to air to discourage anaerobic bacteria growth. In another embodiment, the flexible strip is waterproof, such that, for example a user of the device could shower or bathe, or otherwise get their skin wet without exposing the light-emitting assembly 20 to water. The flexible strip 10 can be formed from materials suitable to be in contact with the skin, which are known in the art, and can include, for example, a woven fabric, polymer (e.g., PVC, polyethylene, or polyurethane), or latex strip. At least a portion of the bottom surface 16 of the flexible attachment strip can include an adhesive configured to secure the device 100 to the skin surface. Suitable adhesives for contacting the skin are known in the art and can include, for example, acrylics, silicones, polyvinyl ethers, synthetic rubbers, and vinyl resins. According to further embodiment, the flexible strip 10 can include one or more fasteners that can be configured to secure the device 100 to the skin by wrapping the strip 10 around the skin and attaching the fasteners to a portion of the flexible strip 10 (e.g., Velcro or serrated clips).

The purpose of the moistening band 50 is to limit infection and the formation of scabbing in and around the wound region. The moistening band 50 maintains an appropriate level of wetness at the wound site and also reduces the evaporation of exudate from the wound. According to one embodiment, the moistening band 50 is transparent. In another embodiment, the moistening band 50 has a transmittance range of about 20% to about 90% of the emitted light from the light-emitting assembly 20. In a further embodiment, the moistening band 50 is a hydrocolloid mass with a polyurethane film.

The flexible device 100 includes a light-emitting assembly 20 including at least one organic light-emitting diode 22 (OLED) and at least one emission modifier 36. According to one embodiment, the light emitting assembly 20 can be disposed between the top surface 54 of the moistening band 50 and the bottom surface 16 of the attachments strip 10. A particular advantage of the device 100, relates to flexibility and energy use of the light-emitting assembly 20. OLEDs have relatively low heat generation and high flexibility as compared to other light sources utilized in phototherapy, which is an advantage with respect to safe and effective attachment to the skin. Additionally, the light-emitting assembly 20 can produce variable wavelengths, which allows for customization of treatment protocol depending upon the nature of the wound.

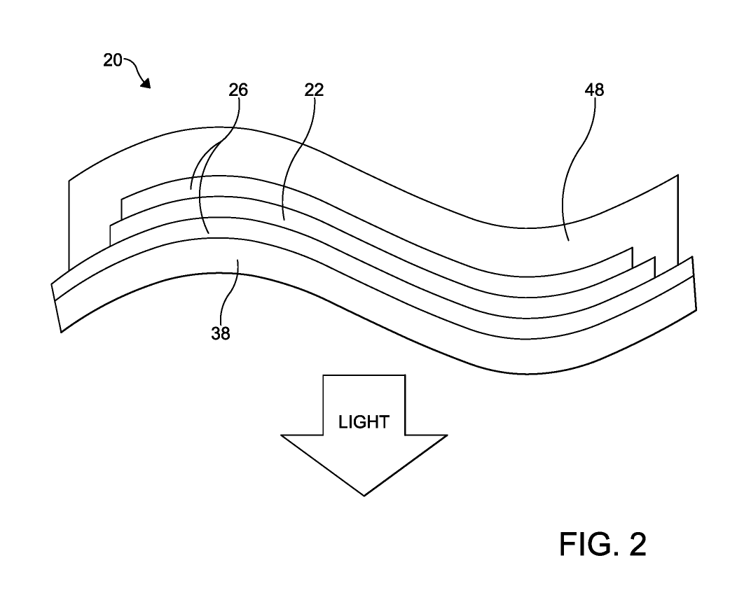

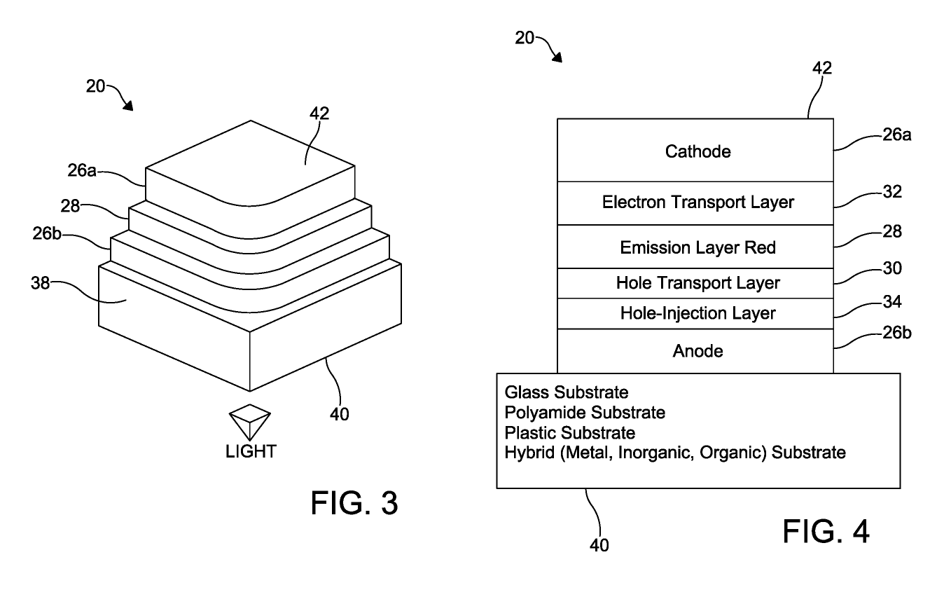

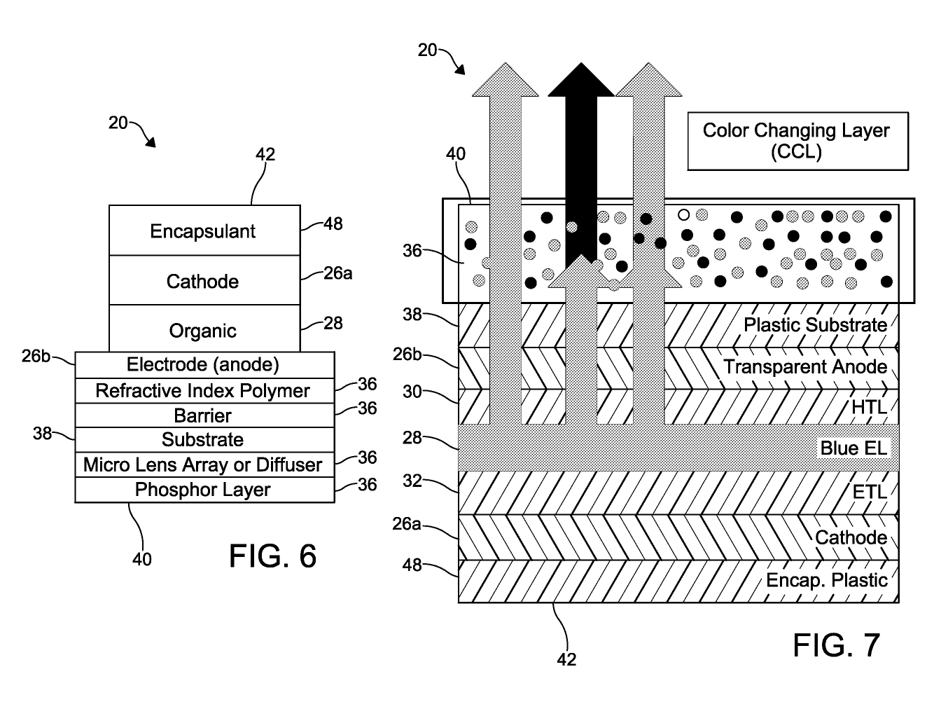

Referring to FIGS. 2-7, the light-emitting assembly 20 includes a bottom surface 40 facing the skin surface and an oppositely disposed top surface 42. According to one embodiment, light-emitting assembly 20 includes an OLED 22 deposited on a substrate 38 and covered with a thin-film encapsulant 48. Thin film encapsulant materials can provide a moisture and/or oxygen barrier for the light-emitting assembly 20 to protect it from degradation. The encapsulant 48 may be composed of organic or inorganic materials. For example, the encapsulant 48 may be made of metal foils, silicone, epoxy, glass, plastic or other materials. The encapsulant 48 is preferably transparent or translucent. According to one embodiment, the encapsulant 48 has a water vapor transmission rate in the range of about 10⁻² to about 10⁻⁶ g/m² per day, for example about 10⁻⁵ to about 10⁻⁶ g/m² per day. As used herein, “g/m²” means gram/meter squared.

According to one embodiment, the substrate 38 has a thickness of less than about 100 μm (as used herein, “μm” means micrometer or micron). Substrate materials suitable for OLEDs and light-emitting assemblies are known in the art, and can include for example, metals, glasses, polyetherimides, polyimides, plastics, fiber reinforced polymers, and can also include hybrid compositions that include a blend of metals, inorganic compositions and organic compositions. According to one embodiment, the substrate 38 is transparent.

The OLED 22 of the light-emitting assembly 20 includes, according to one embodiment, two electrodes 26 with an organic emissive layer 28 disposed between electrodes 26. According to one embodiment, OLED 22 includes an emissive layer 28, a cathode 26a, in contact with, and disposed above the emissive layer 28, and an anode 26b, disposed below, and in contact with the emissive layer 28. Suitable materials for OLED cathodes 26a are known in the art and can included aluminum or aluminum containing compounds. Additionally, cathodes 26a can be formed from other metals such as barium or calcium and contain an aluminum capping layer to avoid degradation. Suitable materials for anodes 26b are also known in the art. In certain embodiments, particularly where the anode 26b is disposed at the light-emitting side of OLED 22, it is desirable for the anode 26b to be formed from a transparent material. According to one embodiment, anode 26b is formed from indium tin oxide (ITO), silver nano-wire, or poly(3,4-ethylenedioxythiophene) (PEDOT).

The OLED 22, according to one embodiment, emits light in a wavelength range of about 410 nm to about 700 nm. According to further embodiment, OLED 22 emits light in a wavelength range of about 410 nm to about 550 nm, and in a still further embodiment, OLED 22 emits light in a wavelength range of about 550 nm to about 700 nm. As used herein, “nm” means nanometer.

According to one embodiment OLED 22 has a single emissive layer 28. The single emissive layer 28 can produce a single color of light, such as for example, blue light, green light, or red light. According to one embodiment, the OLED 22 emits blue light from a blue emissive layer 28, and according to another embodiment, OLED 22 emits red light from a red emissive layer 28. According to another embodiment, OLED 22 can include multiple emissive layers 28, where each layer is configured to emit a separate color of light.

Materials suitable for forming the emissive layer 28 are known in the art and commonly include iridium complexes. Particular iridium complexes can produce light within certain defined wavelengths. Suitable iridium complex materials for a red emissive layer 28 for producing red light can include, for example, Ir(btp)₂(acac), Ir(piq)₂(acac), Ir(piq)₃, Ir(DBQ)₂(acac), Ir(MDQ)₂(acac), Ir(C8piq)₃, Ir(4F5mpiq)₃, Ir(C4-piq)₃, Ir(BPPa)₃, (piq)₂Ir(PO), (nazo)₂Ir(PO), (piq)Ir(PO)₂, (nazo)Ir(PO)₂, (Et-Cvz-PhQ)₂Ir(pic), (EO-Cvz-PhQ)₂Ir(picN-O), (EO-Cvz-PhQ)₂Ir(pic), Ir(phq)₃, Ir(phq)₂acac, Ir(piq)₂acac, and Ir(dbfiq)₂(bdbp). Suitable materials for forming a blue emissive layer can include, for example, Flrpic and 1,3-Bis(carbazol-9-yl)benzene. Suitable materials for forming a green emissive layer can include, for example, Ir(ppy)₃, Ir(ppy)₂(acac), Be(pp)₂.

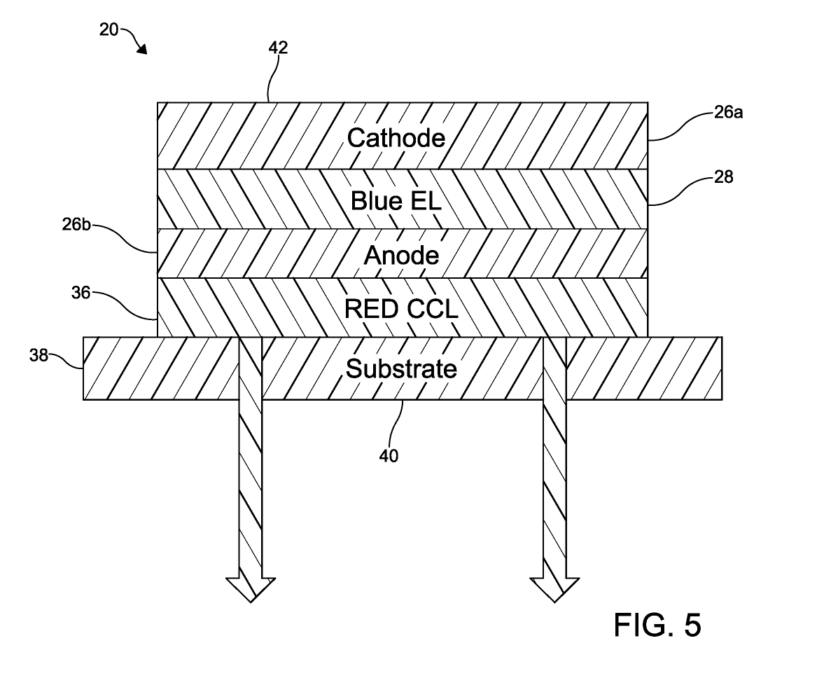

Referring to FIGS. 5-7, the light-emitting assembly 20 includes, according to certain embodiments, at least one emission modifier 36. Emission modifier 36 is a composition or structure that can modify the light emission properties of the light emitted from the source OLED 22. Such properties can include, but are not limited to, transmission intensity, wavelength transmission, and range of transmitted wavelengths. According to one embodiment, the emission modifier 36 is a color conversion layer (CCL), a Bragg reflector, a microlens array, or a combination of thereof. In certain embodiments, the emission modifier 36 is disposed between the substrate 38 and the surface of the skin. In other embodiments, the emission modifier 36 is disposed between the emissive layer 28 and the substrate 38. In still other embodiments, one or more emission modifiers 36 can be disposed both between the substrate 38 and the surface of the skin, and between the emissive layer 28 and the substrate 38.

According to one embodiment, the emission modifier 36 can broaden the wavelength range of light emitted from OLED 22 such that the light-emitting assembly 20 emits light over a greater range of wavelength than the OLED wavelength range. According to another embodiment, the emission modifier 36 can narrow the wavelength range of light emitted from OLED 22 such that the light-emitting assembly 20 emits light over a narrower range of wavelength than the OLED wavelength range. According to another embodiment, the emission modifier 36 can shift the wavelength range of light emitted from OLED 22 such that the light-emitting assembly 20 emits light over a range of wavelength that is different than the OLED wavelength range. For example, in one embodiment, the wavelength range of the light emitted from the light-emitting assembly does not include wavelengths of the wavelength range of the light emitted from the OLED. It should be appreciated that in embodiments where the emission modifier 36 shifts the wavelength range of the light emitted from the OLED 22, the emission modifier 36 can also narrow or broaden the wavelength range as well. In a still further embodiment, the emission modifier 36 can increase, or decrease, the intensity of the light emitted from OLED 22, such that the light emitting assembly 20 emits light in an intensity that is greater than, or less than, the intensity of the light emitted from OLED 22.

According to one embodiment, the emission modifier 36 is a color conversion layer (CCL). The color conversion layer is arranged to receive light or radiation from the OLED 22. According to one embodiment, the color conversion layer is configured to convert at least a portion of the light emitted from the OLED 22 to a different color.

The emission modifier 36 may comprise a film of fluorescent or phosphorescent material (commonly known in the art as a “phosphor”) which efficiently absorbs higher energy photons (e.g. blue light and/or yellow light) and reemits photons at lower energy (e.g. at green and/or red light) depending on the materials used. That is, the color conversion layer may absorb light emitted by OLED 22 and reemit the light (or segments of the wavelengths of the emission spectrum of the light) from the light-emitting assembly 20 at a longer wavelength.

In some aspects, the light-emitting assembly 20 may include more than one color conversion layer. The emission modifier 36 can include a film or layer of color conversion material that is configured to convert at least some of the light emitted by the OLED 22 into light having a different wavelength. For example, the color conversion layer may include a layer of material that is configured to convert the light emitted by the OLED 22 to a higher or lower wavelength. In one aspect of the disclosure, the color conversion material is a phosphor material. For example, if the OLED 22 emits blue light in the blue spectral range of 450-490 nm, then the color conversion layer may contain a layer of phosphor material for converting some of this radiation to a different spectral range. Preferably, the phosphor material is configured to convert most or all of the radiation from the OLED 22 to the desired spectral range. Phosphor materials suitable for this purpose are generally known in the art and may include, but are not limited to yttrium aluminum garnet (YAG) phosphors. Certain phosphor compounds can provide specific color emission, such as, for example, Lu₃Al₅Al₁₀O₁₂:Ce (LuAG:Ce) for a green color, Y₃Al₅O₁₂:Ce (YAG:Ce) for yellow color, and Sr₂Si₅N₈:Eu or CaAlSiN₃:Eu for red.

The phosphor material is typically in the form of a powder. The phosphor powder may be composed of phosphor particles, phosphor microparticles, phosphor nanoparticles or combinations thereof. The phosphor particles or phosphor microparticles may have an average diameter that ranges in size from 1 micron to 100 microns. In one aspect of the present disclosure, the average diameter of the phosphor particles is less than 50 microns. In another aspect of the present disclosure, the average diameter of the phosphor particles is less than 20 microns. In yet another aspect of the present disclosure, the average diameter of the phosphor particles is less than 10 microns. In yet another aspect of the present disclosure, the average diameter of the phosphor nanoparticles used in the phosphor powder ranges from 10 nm to 900 nm. The size of the phosphor particles is generally selected based on the desired thickness of the color conversion layer and/or the overall thickness of the color conversion layer.

The phosphor powder may be dispersed in a binder material that is useful in forming a film or a sheet. A uniform distribution of the phosphor powder in the binder material and throughout the color conversion layer is generally preferred to achieve a consistent color quality of light from the light-emitting device. More uniform color quality and brightness.

The binder material may be organic or inorganic. In one aspect of the present disclosure the binder material is transparent or translucent. In another aspect of the present disclosure, the binder may be a UV-curable binder. The binder material may also be curable thermally. Examples of binder materials suitable for use with the phosphor material may include, but are not limited to silicone resin, epoxy resin, polyallylate resin, PET modified polyallylate resin, polycarbonate resin (PC), cyclic olefin, a polyethylene terephthalate resin (PET), polymethylmethacrylate resin (PMMA), a polypropylene resin (PP), modified acryl resin, polystyrene resin (PE), and acrylonitrile-styrene copolymer resin (AS). The binder material may include combinations or mixtures of these and/or other suitable materials. For example, additives may be added to the binder material to improve or alter certain properties of the color conversion layer as needed.

According to another embodiment, the emission modifier 36 is a Bragg reflector. Distributed Bragg reflectors (DBR) are known in the art and are intended to function by reflecting a broad spectrum of light and transmit only a narrow range of wavelengths. DBR structures typically include one or more stacks of alternating layers of a first high refractive index material and a second low refractive index material. In certain embodiments, DBRs can be a mirror structure that includes an alternating sequence of layers of two different optical materials. One such design is a quarter-wave mirror, in which each optical layer thickness corresponds to one quarter of the wavelength for which the mirror is designed. Conventionally, fabrication of DBRs often entails stacking varying inorganic dielectric thin films, such as TiO₂/SiO₂ and Al₂O₃/HfO₂ bilayers, on plastic substrates. The use of DBRs having these inorganic bilayers is advantageous because they can provide wide bandwidth and high reflectivity with only a few pairs of bilayers.

Referring to FIG. 7, a particular embodiment of a light-emitting assembly 20 is shown including a hybrid OLED 22 and is formed using a color conversion layer (CCL) as emission modifier 36 with a blue emissive layer 28 to produce light across the full wavelength spectrum of visible light. The emission modifier 36 (e.g., CCL) contains a phosphor material that scatters a portion of the light from the blue emissive layer 28. The combination of the light emitted from the emission modifier 36 and the unabsorbed light from the blue emissive layer 28 produces white light.

According to one embodiment, the light-emitting assembly 20 emits light in a wavelength range of about 400 nm to about 900 nm, or from about 410 nm to about 700 nm, or from about 410 nm to about 550 nm, or from about 550 nm to about 700 nm.

According to a further embodiment, flexible device 100 can include one or more heat-dissipating films. The heat-dissipating film can include a thermally conductive material. Thermally conductive materials are known in the art and can include, for example, metal salts, metal oxides, metal hydroxides, for example, aluminum oxide hydroxides including boehmite γ-AlO(OH), diaspore α-AlO(OH), and gibbsite Al(OH)₃, or magnesium hydroxide Mg(OH)₂; oxides such as calcium oxide CaO, magnesium oxide MgO, zinc oxide ZnO, titanium dioxide TiO₂, tin dioxide SnO₂, chromium oxides including chromium(II) oxide CrO, chromium(III) oxide Cr₂O₃, chromium dioxide (chromium(IV) oxide) CrO₂, chromium trioxide (chromium(VI) oxide) CrO₃, and chromium(VI) oxide peroxide CrO₅, barium oxide BaO, silicon dioxide SiO₂, zirconium dioxide ZrO₂, magnesium aluminate MgO*Al₂O₃, aluminum oxide Al₂O₃, or beryllium oxide BeO; carbonates such as calcium carbonate CaCO₃, or calcium magnesium carbonate (Dolomite) CaMg(CO₃)₂; sulfates such as barium sulfate BaSO₄, or calcium sulfate CaSO₄; silicates such as zinc silicate, mica, glass beads/fibers, calcium silicate (wollastonite) CaSiO₃, magnesium silicate (talc) H₂Mg₃(SiO₃)₄/Mg₃Si₄O₁₀(OH)₂, or clay; nitrides such as aluminum nitride AlN, boron nitride BN, aluminum oxynitride AlON, magnesium silicon nitride MgSiN₂, or silicon nitride Si₃N₄; phosphides such as aluminum phosphide AlP, or boron phosphide BP; sulfides such as cadmium sulfide CdS or zinc sulfide ZnS; and, carbides such as aluminum carbide Al₄C₃, or silicon carbide SiC, or combinations or mixtures thereof.

In a further embodiment of the disclosure, and referring to FIGS. 1A-C, flexible device 100 can include one or more sensors 70 disposed along the bottom surface 16 of the flexible attachment strip 10, an antenna 18, for example a near field antenna (NFC) connected to the flexible strip 10, a power source 60, for example a thin film or coin cell battery, connected to the flexible strip 10, and a flexible printed circuit board (F/PCB) 80, including a one or more integrated circuit chips for communication and memory, connected to the flexible strip 10. A power switch 66 can be optionally included to provide the user with the ability to power on or off the flexible device 100.

Sensors 70 can be utilized to acquire treatment data from the wound site and transmit that data through the F/PCB 80, such that the data can be transmitted through antenna 18. Sensors 70, according to one embodiment can detect one or more of the following bodily conditions for the purpose of collecting treatment data: body temperature, blood sugar levels, heart rate, blood pressure, blood oxygen levels, and electrocardiology information.

According to one embodiment, flexible device 100 has wireless communication capabilities and is capable of sending and receiving treatment protocol data regarding conditions at the wound site and treatment parameters for the light-emitting assembly. The flexible device 100 therefore can gather information from the sensors 70 included and check the status of patient health and the status of the wound site by utilizing a remote medical service from this information. The device 100, according to one embodiment, is configured to connect to medical software at an external terminal such as a smart phone, desktop, notebook, tablet, etc.

Further according to the present disclosure, a method for therapeutic treatment of a wound is disclosed including determining a treatment protocol; attaching the flexible device 100 to a surface of the skin, and activating the light-emitting assembly 20 to emit light having a wavelength range and intensity according to the treatment protocol.

The method of therapeutic treatment can, according to another embodiment, include determining a first treatment protocol; attaching the flexible device 100 to a surface of the skin; activating the light-emitting assembly to emit light having a wavelength range and intensity according to the first treatment protocol; acquiring treatment data of the first protocol through the one or more sensors; transmitting the treatment data from the one or more sensors of the device to an external terminal; comparing treatment data from the first protocol treatment with a treatment database; determining an adjustment from the first treatment protocol to a second treatment protocol; transmitting instructions from the external terminal to the communication microchip through the antenna of the device for the second protocol; and, activating the light-emitting assembly to emit light having a wavelength range and intensity according to the second treatment protocol.

Aspects

The present disclosure includes the following aspects:

Aspect 1. A flexible device for phototherapy at a skin surface of a user, the flexible device comprising: a flexible attachment strip configured to secure the device to the skin surface, the flexible attachment strip having a bottom surface facing the skin surface and an oppositely disposed top surface; a moistening band attached to the bottom surface of the attachment strip, the moistening band having a bottom surface facing the skin surface and an oppositely disposed top surface, the bottom surface configured to contact the skin surface; and, a light-emitting assembly comprising at least one organic light-emitting diode (OLED) and at least one emission modifier, wherein the light-emitting assembly has a bottom surface facing the skin surface and an oppositely disposed top surface, the light emitting assembly disposed between the top surface of the moistening band and the bottom surface of the attachments strip; wherein at least a portion of the bottom surface of the flexible attachment strip is configured to contact the skin surface.

Aspect 2. The device of aspect 1, wherein the OLED comprises a blue emissive layer that emits light in a wavelength range of about 410 nm to about 550 nm.

Aspect 3. The device of aspect 1, wherein the OLED comprises a red emissive layer that emits light in a wavelength range of about 550 nm to about 700 nm.

Aspect 4. The device of aspect 1, wherein the light-emitting assembly emits light in a wavelength range of about 410 nm to about 700 nm.

Aspect 5. The device of aspect 4, wherein the light-emitting assembly emits light in a wavelength range of about 410 nm to about 550 nm.

Aspect 6. The device of aspect 4, wherein the light-emitting assembly emits light in a wavelength range of about 550 nm to about 700 nm.

Aspect 7. The device of any of the preceding aspects, wherein the emission modifier comprises a color conversion layer, a Bragg reflector film, a microlens array, or any combination thereof.

Aspect 8. The device of any of the preceding aspects, wherein the emission modifier reduces the intensity of light emitted from the OLED.

Aspect 9. The device of aspect 7, wherein the emission modifier changes the wavelength range of the light emitted from the OLED.

Aspect 10. The device of aspect 9, wherein the emission modifier broadens the wavelength range of the light emitted from the OLED.

Aspect 11. The device of aspect 9, wherein the emission modifier narrows the wavelength range of the light emitted from the OLED.

Aspect 12. The device of aspect 9, wherein the wavelength range of the light emitted from the light-emitting assembly does not include wavelengths of the wavelength range of the light emitted from the OLED.

Aspect 13. The device of any one of the preceding aspects, further comprising a heat dissipation material.

Aspect 14. The device of aspect 13, wherein the heat dissipation material includes a thermally conductive film material.

Aspect 15. The device of any of the preceding aspects wherein the moistening band comprises one or materials such that the moistening band has a transmittance range of about 20% to about 90% of the emitted light from the light-emitting assembly.

Aspect 16. The device of any of the preceding aspects, wherein at least a portion of the bottom surface of the flexible attachment strip includes an adhesive configured to secure the device to the skin surface.

Aspect 17. The device of any one of the preceding aspects, wherein the flexible attachment strip includes one or more fasteners.

Aspect 18. A flexible device for phototherapy at a skin surface of a user, the flexible device comprising: a flexible attachment strip configured to secure the device to the skin surface, the flexible attachment strip having a bottom surface facing the skin surface and an oppositely disposed top surface, wherein at least a portion of the bottom surface of the flexible attachment strip is configured to contact the skin surface; a moistening band attached to the bottom surface of the attachment strip, the moistening band having a bottom surface facing the skin surface and an oppositely disposed top surface, the bottom surface configured to contact the skin surface; a light-emitting assembly comprising at least one organic light-emitting diode (OLED) and at least one emission modifier, wherein the light-emitting assembly has a bottom surface facing the skin surface and an oppositely disposed top surface, the light emitting assembly disposed between the top surface of the moistening band and the bottom surface of the attachments strip; one or more sensors disposed along the bottom surface of the flexible attachment strip; a near field communication (NFC) antenna connected to the flexible attachment strip; a battery connected to the flexible attachments strip; and, a flexible printed circuit board (FBCB) including a communication microchip and a memory microchip connected to the flexible attachment strip.

Aspect 19. A method for phototherapeutic treatment of a wound comprising: determining a treatment protocol; attaching the flexible device of any one of aspects 1-17, and activating the light-emitting assembly to emit light having a wavelength range and intensity according to the treatment protocol.

Aspect 20. A method for phototherapeutic treatment of a wound comprising: determining a first treatment protocol; attaching the flexible device of aspect 18; activating the light-emitting assembly to emit light having a wavelength range and intensity according to the first treatment protocol; acquiring treatment data of the first protocol through the one or more sensors; transmitting the treatment data from the one or more sensors of the device to an external terminal; comparing treatment data from the first protocol treatment with a treatment database; determining an adjustment from the first treatment protocol to a second treatment protocol; transmitting instructions from the external terminal to the communication microchip through the antenna of the device for the second protocol; and activating the light-emitting assembly to emit light having a wavelength range and intensity according to the second treatment protocol.

What is claimed:

1. A flexible device for phototherapy at a skin surface of a user, the flexible device comprising:
a flexible attachment strip configured to secure the device to the skin surface, the flexible attachment strip having a bottom surface facing the skin surface and an oppositely disposed top surface;
a moistening band attached to the bottom surface of the attachment strip, the moistening band having a bottom surface facing the skin surface and an oppositely disposed top surface, the bottom surface configured to contact the skin surface; and
a light-emitting assembly comprising at least one organic light-emitting diode (OLED) and at least one emission modifier, wherein the light-emitting assembly has a bottom surface facing the skin surface and an oppositely disposed top surface, the light emitting assembly disposed between the top surface of the moistening band and the bottom surface of the attachments strip;
wherein at least a portion of the bottom surface of the flexible attachment strip is configured to contact the skin surface.

2. The device of claim 1, wherein the OLED comprises a blue emissive layer that emits light in a wavelength range of about 410 nm to about 550 nm.

3. The device of claim 1, wherein the OLED comprises a red emissive layer that emits light in a wavelength range of about 550 nm to about 700 nm.

4. The device of claim 1, wherein the light-emitting assembly emits light in a wavelength range of about 410 nm to about 700 nm.

5. The device of claim 4, wherein the light-emitting assembly emits light in a wavelength range of about 410 nm to about 550 nm.

6. The device of claim 4, wherein the light-emitting assembly emits light in a wavelength range of about 550 nm to about 700 nm.

7. The device of claim 1, wherein the emission modifier comprises a color conversion layer, a Bragg reflector film, a microlens array, or any combination thereof.

8. The device of claim 1, wherein the emission modifier reduces the intensity of light emitted from the OLED.

9. The device of claim 7, wherein the emission modifier changes the wavelength range of the light emitted from the OLED.

10. The device of claim 9, wherein the emission modifier broadens the wavelength range of the light emitted from the OLED.

11. The device of claim 9, wherein the emission modifier narrows the wavelength range of the light emitted from the OLED.

12. The device of claim 9, wherein the wavelength range of the light emitted from the light-emitting assembly does not include wavelengths of the wavelength range of the light emitted from the OLED.

13. The device of claim 1, further comprising a heat dissipation material.

14. The device of claim 13, wherein the heat dissipation material includes a thermally conductive film material.

15. The device of claim 1, wherein the moistening band comprises one or more materials such that the moistening band has a transmittance range of about 20% to about 90% of the emitted light from the light-emitting assembly.

16. The device of claim 1, wherein at least a portion of the bottom surface of the flexible attachment strip includes an adhesive configured to secure the device to the skin surface.

17. The device of claim 1, wherein the flexible attachment strip includes one or more fasteners.

18. A flexible device for phototherapy at a skin surface of a user, the flexible device comprising:
a flexible attachment strip configured to secure the device to the skin surface, the flexible attachment strip having a bottom surface facing the skin surface and an oppositely disposed top surface, wherein at least a portion of the bottom surface of the flexible attachment strip is configured to contact the skin surface;
a moistening band attached to the bottom surface of the attachment strip, the moistening band having a bottom surface facing the skin surface and an oppositely disposed top surface, the bottom surface configured to contact the skin surface;
a light-emitting assembly comprising at least one organic light-emitting diode (OLED) and at least one emission modifier, wherein the light-emitting assembly has a bottom surface facing the skin surface and an oppositely disposed top surface, the light emitting assembly disposed between the top surface of the moistening band and the bottom surface of the attachments strip;
one or more sensors disposed along the bottom surface of the flexible attachment strip;
a near field communication (NFC) antenna connected to the flexible attachment strip;
a battery connected to the flexible attachments strip; and,
a flexible printed circuit board (FBCB) including a communication microchip and a memory microchip connected to the flexible attachment strip.

19. A method for phototherapeutic treatment of a wound comprising:
determining a treatment protocol;
attaching the flexible device of claim 1, and
activating the light-emitting assembly to emit light having a wavelength range and intensity according to the treatment protocol.

20. A method for phototherapeutic treatment of a wound comprising:
determining a first treatment protocol;
attaching the flexible device of claim 18;
activating the light-emitting assembly to emit light having a wavelength range and intensity according to the first treatment protocol;
acquiring treatment data of the first protocol through the one or more sensors;
transmitting the treatment data from the one or more sensors of the device to an external terminal;
comparing treatment data from the first protocol treatment with a treatment database;
determining an adjustment from the first treatment protocol to a second treatment protocol;
transmitting instructions from the external terminal to the communication microchip through the antenna of the device for the second protocol; and
activating the light-emitting assembly to emit light having a wavelength range and intensity according to the second treatment protocol.
